(19) United States
(12) Patent Application Publication
(10) Pub. No.: US 2003/0187334 A1
(43) Pub. Date: Oct. 2, 2003

(54) Uterine and vaginal vault manipulator

(75) Inventor: Nicholas Biswas, Winston Hills (AU)

Correspondence Address: LADAS & PARRY, 26 WEST 61ST STREET, NEW YORK, NY, 10023, US

(73) Assignee: FREESPIRIT UNLIMITED PTY LTD.

(21) Appl. No.: 10/361,785

(22) Filed: Feb. 10, 2003

(30) Foreign Application Priority Data
Feb. 11, 2002 (AU) PS 0412
Jun. 14, 2002 (AU) PS 2955

(51) Int. Cl. A61B 1/32

(52) U.S. Cl. 600/227; 600/235

(57) ABSTRACT

A surgical instrument 10 to aid a medical practitioner to manipulate a patients uterus and vaginal vault, the instrument 10 includes a shaft 12 that is inserted through the patient's vagina and cervix so as to protrude into the uterus. A hollow member 19 is sealingly engaged with the shaft 12 and extends therefrom so as to engage the vaginal wall to sealingly connect the shaft 12 with the vaginal wall. This enables a surgeon to cut the vaginal wall surrounding the cervix while delivering a gas under pressure to the body cavity surrounding the uterus.

### TECHNICAL FIELD

The present invention relates to surgical instruments and more particularly to an instrument to aid in the manipulation of the uterus and vaginal vault.

### BACKGROUND OF THE INVENTION

Some surgical procedures, such as hysterectomies, are facilitated by the use of surgical instruments that aids the surgeon in manipulating and positioning the uterus and vaginal vault. One previous instrument includes a conical member with an external thread that projects into the cervix but does not protrude therefrom into the uterus.

It is not uncommon during hysterectomies for the surgeon to deliver a gas under pressure to the body cavity surrounding the uterus to free the area around the uterus. A clamp may also be employed in the procedure to engage the external surface of the cervix for the purposes of manipulating the uterus and vaginal vault. The purpose of the manipulation is to expose the vaginal wall adjacent the cervix to aid the surgeon in cutting the vaginal wall. The purpose of the manipulation may also be for the division of adhesions, endometrosis and similar conditions.

During the above discussed procedure, upon cutting the vaginal wall, the gas under pressure escapes. Accordingly, the areas surrounding the uterus is no longer clear. A further disadvantage is that the abovementioned clamp can frequently not effectively engage the cervix and therefore, may not enable the surgeon to manipulate the uterus and vaginal vault to the extent required.

### OBJECT OF THE INVENTION

It is the object of the present invention to overcome or substantially ameliorate at least one of the above disadvantages.

### SUMMARY OF THE INVENTION

There is disclosed herein a surgical instrument to aid a medical practitioner to manipulate a patient's uterus and vaginal vault, said instrument including:

a shaft to pass through the vagina and to enter the cervix of the patient, said shaft having protrusions to aid the shaft in securely engaging the cervix; and

a vaginal wall engaging member sealingy mounted on the shaft and to extend therefrom to sealingly engage the vaginal wall surrounding the cervix.

Preferably said projection is a helical ridge extending longitudinally and angularly about said shaft.

Preferably said member is of a hollow frusto conical configuration so as to have a radially larger end to engage the vaginal vault, and a radially smaller end provided with an aperture through which said shaft passes and via which said shaft sealingly engages said member.

Preferably said shaft has a first end to be located within the uterus, and a second end remote from said first end, with said second end being adapted to be manipulated by the medical practitioner.

Preferably said second end is provided with a handle to aid the medical practitioner in moving said shaft.

Preferably said shaft includes a first portion to project through the cervix and into the uterus, and a second portion to be manipulated by the medical practitioner, and wherein said device further includes a hinge connecting the first and second portions.

Preferably said instrument includes lock means to selectively retain the first and second portions in a desired relative position.

Preferably said first portion is of a length so to have an extremity to engage the uterus.

Preferably said instrument includes an expansion member positioned on said first portion, said expansion member having a contracted configuration enabling insertion into the uterus, and an expanded configuration facilitating engagement with the uterus and therefore aiding said manipulation.

Preferably the hinge includes a yoke providing a pair of spaced arms, and a pin extending between the arms, and a projection providing a slot through which the pin passes, the projection being located between the arms so that the pin may travel longitudinally of the slot.

Preferably said projection is provided on said first portion and said yoke on said second portion.

In a further preferred form the hinge includes an aperture in said first portion, a shaft extending from said second portion through said aperture to provide a pivot axis generally normal to the shaft.

There is further disclosed herein, in combination with the above surgical instrument, a stabilising device to aid in maintaining the shaft in a desired orientation and position relative to the patient, said stabilising device including:

a pair of leg engaging members, each leg engaging member being adapted to be secured to a respective leg of the patient; and

a bridge extending between the leg engaging members, the bridge including shaft engaging means to engage the shaft to aid in maintaining the orientation and position of the shaft.

Preferably, the shaft engaging means includes a shaft engaging portion operable to selectively engage the shaft.

### BRIEF DESCRIPTION OF THE DRAWINGS

A preferred form of the present invention will now be described by way of example with reference to the accompanying drawings wherein:

FIG. 1 is a schematic sectioned side elevation of an instrument to aid a medical practitioner to manipulate a patient's uterus and vaginal vault;

FIG. 2 is a schematic part side elevation of a modification of the instrument of FIG. 1;

FIG. 3 is a schematic side elevation of the instrument of FIG. 2;

FIG. 4 is a further schematic side elevation of the instrument of FIG. 2;

FIG. 5 is a schematic part section side elevation of a modification of the instrument of FIG. 1;

FIG. 6 is a schematic side elevation of a modification of the instrument as shown in FIG. 4;

FIG. 7 is a schematic elevation of a modification of the hinge illustrated in FIG. 6;

FIG. 8 is a schematic elevation of an alternative hinge to that shown in FIGS. 6 and 7;

FIG. 9 is a schematic front elevation of a stabilising device to be employed with the instrument of FIGS. 1 to 8; and

FIG. 10 is a schematic elevation of a portion of the device of FIG. 9.

### DETAILED DESCRIPTION OF THE PREFERRED EMBODIMENTS

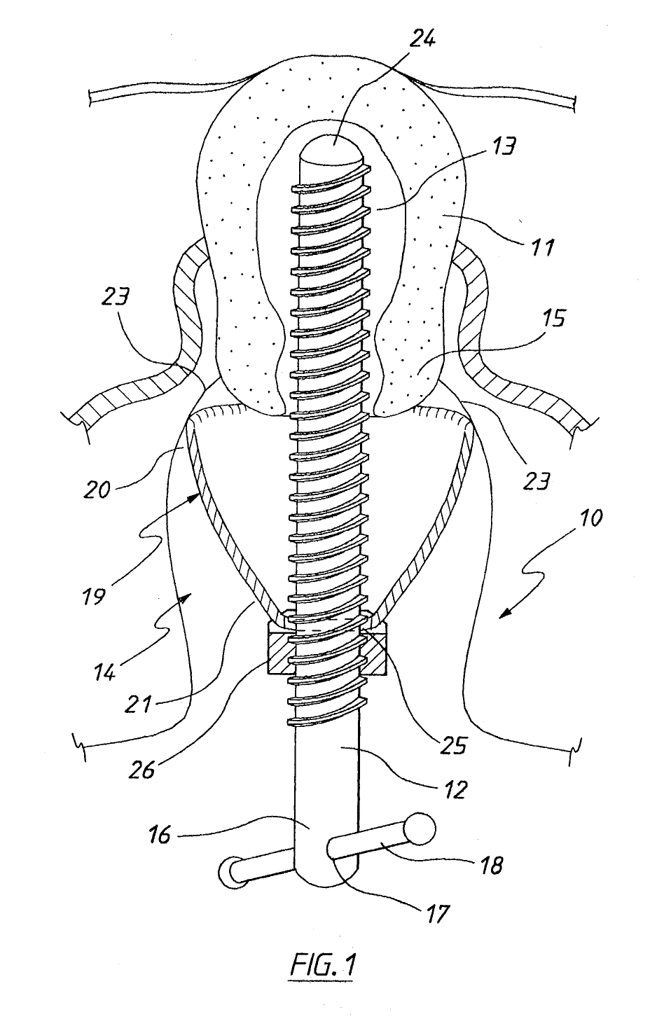

In FIG. 1 of the accompanying drawings, there is schematically depicted an instrument 10 to engage within the uterus 11 of a patient.

The instrument 10 includes a shaft 12 which has a helical projection 13 extending angularly about and longitudinally of the shaft 12. The shaft 12 enters the uterus 11 by the medical practitioner inserting the shaft 12 through the patient's vagina 14 and cervix 15 so as to protrude in the uterus 11. More particularly, the helical projection 13 is shaped to securely engage within the cervix 15 and uterine body so that the medical practitioner may grip the distal end portion 16 of the shaft 12, to move the shaft 12 and therefore manipulate the uterus 11. The distal end 16 is provided with an aperture 17 through which there slidably passes a rod 18 (handle) that may be gripped by the medical practitioner to apply torque to the shaft 12 to cause rotation thereof.

Sealingly engaged with the shaft 12 is a vaginal wall engaging member 19 which is hollow and which is of a frusto conical configuration. The member 19 has a radially larger end 20 that engages the vaginal wall to sealingly connect the member 19 thereto. The radially smaller end 21 of the member 19 sealingy engages the shaft 12 so that the cavity 22 is generally sealingly closed. This then enables a surgeon to cut the vaginal wall portion 23 surrounding the cervix 15 without the pressurised gas escaping through the vagina.

The inner end 24 of the shaft 12 is shaped to inhibit penetration of the uterus 11.

The end 21 of the member 19 has an aperture 25 through which the shaft 12 passes. More particularly, the shaft 12 would “threadably” engage the end 21 of the member 19 so that relative rotation between the member 19 and the shaft 12 will cause the member 19 to move axially along the shaft 12. Still further, threaded engagement between the projection 13 and the cervix 15 facilitates insertion since rotation of the shaft 12 will cause longitudinal progression of the shaft 12 relative to the uterus 11.

The shaft 12 may have markings to indicate depth of penetration.

In an alternative embodiment, the member 19 may merely slide along the shaft 12, and be held in position by a nut 26 threadably engaged with the shaft 12.

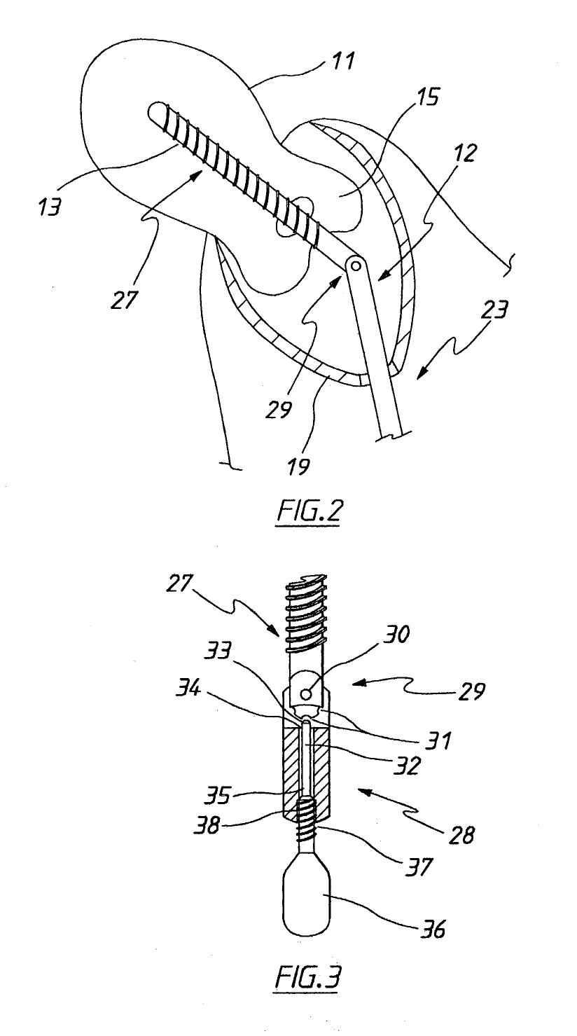

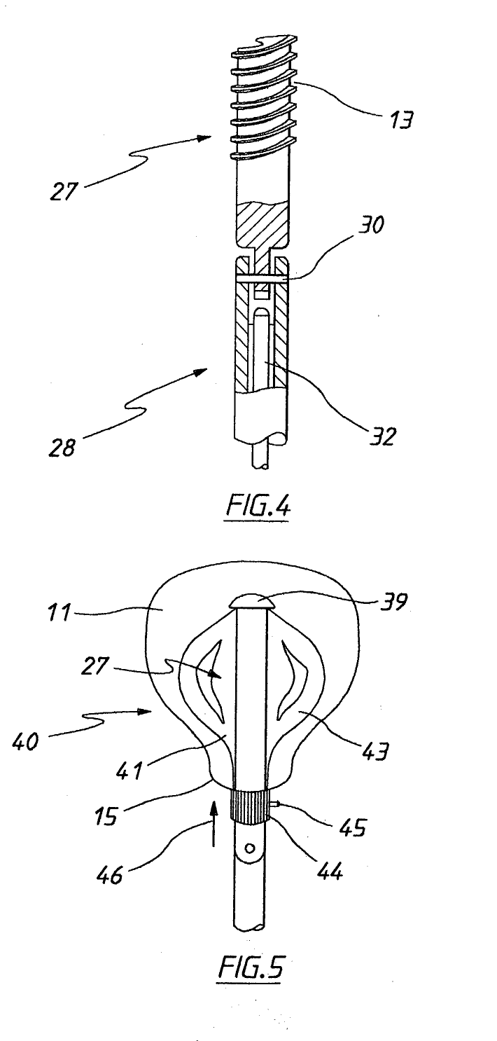

In the embodiment of FIGS. 2 to 4 the instrument 10 of the above described preferred embodiment has been modified so that the shaft 12 has a first portion 27 that projects into the uterus 11, and a second portion 28 that is manipulated by the medical practitioner. The first portion 27 is connected to the second portion 28 by means of a hinge 29 so that the displacement of the second portion 28 may be adjusted relative to the first portion 27. More particularly the hinge 29 includes a hinge pin 30 that provides for pivoting movement between the first portion 27 and second portion 28 about an axis generally normal to the shaft 12. The first portion 27 is provided with the helical projection 13 and has at one extremity notches 31. The second portion 28 is hollow and includes an elongated member 32 having an extremity 33 that is selectively engageable in one of the notches 31. More particularly the elongated member 32 passes through a guide hole 34 and has its other extremity 35 provided with a handle 36 The handle 36 is attached to the elongated member 32 so that rotation of the handle 36 also causes rotation of the elongated member 32 about its longitudinal axis. More particularly, the handle 36 has a threaded portion 37 that is threadably engaged with an aperture 38 in the first portion 28 to cause the above mentioned longitudinal movement. Accordingly, by manipulation of the handle 36 the medical practitioner may angularly adjust the displacement of the second portion 28 relative to the first portion 27. Once a desired position is arrived at the handle 36 is rotated so that the extremity 33 is engaged within one of the notches 31 so that the second portion 28 is locked to the first portion 27,

In the embodiment of FIG. 5 the inner end 24 of the shaft 12 is provided with an enlarged cap 39 that engages the internal surfaces of the uterus 11 to aid in manipulation thereof. More particularly, this embodiment includes an expansion member 40 that is moveable between a contracted figuration allowing insertion of the first portion 27 and the expanded configuration (as depicted) in which the member 40 is expanded to engage within the uterus 11. In the expanded configuration the member 40 aids the impudent 10 in gripping the uterus 11.

The member 40 is an elastically deformable sleeve 41 that is longitudinally slotted so as to have slots 42 separating bands 43. When relaxed, the sleeve 41 is collapsed so that the bands 43 extends generally parallel to the shaft 12. In this contracted configuration the first portion 27 is insertable through the cervix 15. Once inserted the sleeve 14 is compressed so that the bands 43 move radially outward. In this expanded configuration the member 40 aids in gripping the uterus 16 and therefore aids the medical practitioner in manipulating the uterus 11.

The member 40 is resiliently deformed when moved to is expanded configuration. Accordingly, when released it reverts to its contracted configuration for removal.

The member 40 is moved from the contracted configuration to the expanded configuration by means of a collar 44 that is moved longitudinally of the portion 27. The collar 44 retained in position by means of a locking pin 45 that engages the portion 27. When the collar 44 is moved in the direction of the arrow 46 the member 40 is caused to expand. When moved in the opposite direction to the arrow 46 the member 40 is caused to retract.

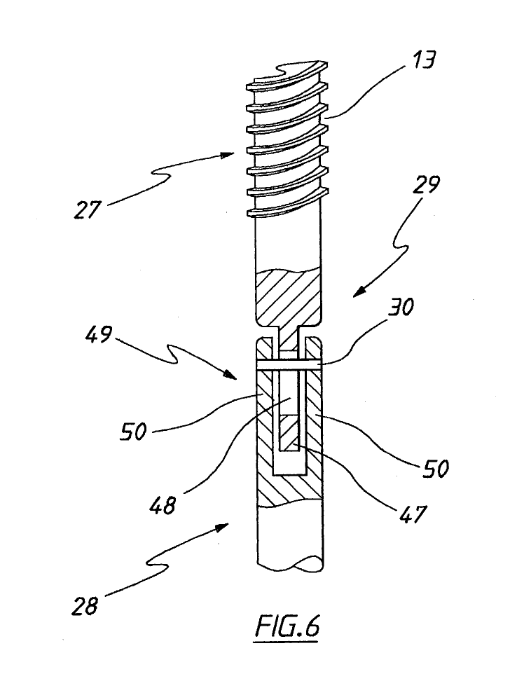

In FIG. 6 a modification of the hinge 29 is depicted. In this embodiment the first portion 27 is provided with a longitudinally extending projection 47 having a longitudinally extending slot 48. The second portion 28 has a yoke 49 providing a pair of spaced arms 50. Extending between the arms 50 is a pin 30 that also passes through the slot 48. The pin 30 is movable along the slot 48 to thereby permit longitudinal displacement between the portions 27 and 28 as well as relative angular movement.

In FIG. 7 there is schematically depicted a modification of the hinge 29 of FIG. 6. In this embodiment the slot 48 extends to a neck 51. The pin 30 is movable through the neck 51 by resilient deflection of the arms 50.

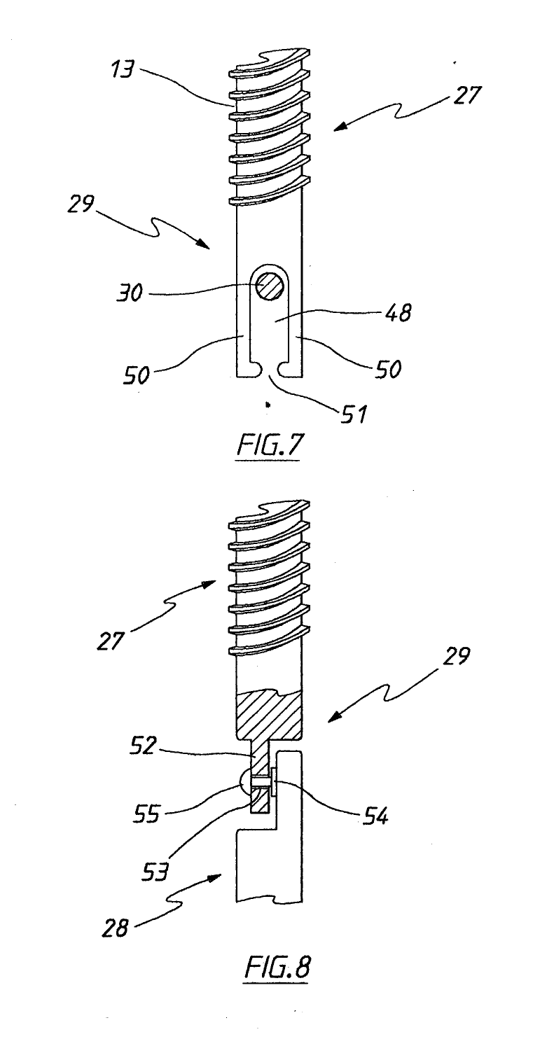

In FIG. 8 a further modification of the hinge 29 is depicted. In this embodiment, the first portion 27 has a flange 52 with an aperture 53. Extending through the aperture 53 is a shaft 54 attached to the second portion 27 The shaft 54 has a head 55 that is resiliently deformed so that it is insertable through the aperture 53.

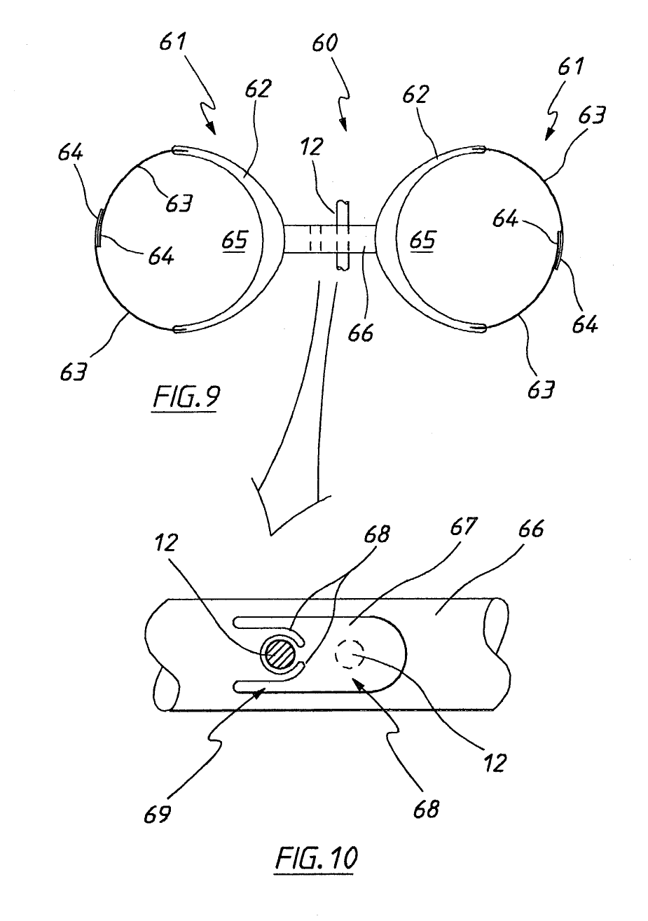

In FIG. 9 there is schematically depicted a stabilisation device 60. The device 60 includes a pair of leg engaging members 61, each member 61 including a generally rigid socket 62 from which a pair of straps 63 extend. The straps 63 have pads 64 of “Velcro” (Registered Trademarks) material so that their position of engagement can be varied, Each socket 62 and associated straps 63 encircle an area 65 through which a patient's leg would pass so that the leg engaging members 61 are secured thereto.

Extending between the leg engaging members 61 is a bridge 66 having an elongated aperture 67 through which the shaft 12 passes. The shaft 12 is movable between a first position 68 at which it is freely movable relative to the bridge 66, and a second position 69 at which it is engaged between a pair of resilient flanges 68 to be retained in position with respect to the bridge 66. More particularly, the shaft 12 would be retained in orientation and positioned relative to the device 60. The flanges 66 are urged into contact with the shaft 12 but are resiliently departed to enable the shaft 12 to be moved to the position 68. When the shaft 12 is located in a desired position it is moved to the position 69 so as to be maintained in that position.

The claims defining the invention are as follows:

1. A surgical instrument to aid a medical practitioner to manipulate a patient's uterus and vaginal vault, said instrument including:
a shaft to pass through the vagina and to enter the cervix of the patient, said shaft having protrusions to aid the shaft in securely engaging the cervix; and
a vaginal wall engaging member sealingly mounted on the shaft and to extend therefrom to sealingly engage the vaginal wall surrounding the cervix.

2. The instrument of claim 1 wherein said projection is a helical ridge extending longitudinally and angularly about said shaft.

3. The instrument of claim 2 wherein said member is of a hollow frusto conical configuration so as to have a radially larger end to engage the vaginal vault, and a radially smaller end provided with an aperture through which said shaft passes and via which said shaft sealingly engages said member.

4. The intent of claim 3 wherein said shaft has a first end to be located within the uterus, and a second end remote from said first end, with said second end being adapted to be manipulated by the medical practitioner.

5. The instrument of claim 3 wherein said second end is provided with a handle to aid the medical practitioner in moving said shaft.

6. The instrument of claim 3 said shaft includes a first portion to project through the cervix and into the uterus, and a second portion to be manipulated by the medical practitioner, and wherein said device further includes a hinge connecting the first and second portions.

7. The instrument of claim 5 further including said instrument includes lock means to selectively retain the first and second portions in a desired relative position.

8. The instrument of claim 6 wherein said first portion is of a length so to have an extremity to engage the uterus.

9. The instrument of any one of claim 7 further including said instrument includes an expansion member positioned on said first portion, said expansion member having a contracted configuration enabling insertion into the uterus, and an expanded configuration facilitating engagement with the uterus and therefore aiding said manipulation.

10. The instrument of any one of claim 7 wherein the hinge includes a yoke providing a pair of spaced arms, and a pin extending between the arms, and a projection providing a slot through which the pin passes, the projection being located between the arms so that the pin may travel longitudinally of the slot.

11. The instrument of claim 10 wherein said projection is provided on said first portion and said yoke on said second portion.

12. The instrument of claim 6 wherein the hinge includes an aperture in said first portion, a shall extending from said second portion through said aperture to provide a pivot axis generally normal to the shaft.

13. In combination the instrument of claim 1 and a stabilising device to aid in maintaining the shaft in a desired orientation and position relative to the patient, said stabilising device including:
a pair of leg engaging members, each leg engaging member being adapted to be secured to a respective leg of the patient; and
a bridge extending between the leg engaging members, the bridge including shaft engaging means to engage the shaft to aid in maintaining the orientation and position of the shaft.

14. The combination of claim 13 wherein the so engaging means includes a shaft engaging portion operable to selectively engage the shaft.
